(19) United States
(12) Patent Application Publication
(10) Pub. No.: US 2022/0339324 A1
(43) Pub. Date: Oct. 27, 2022

(54) ACELLULAR ARTIFICIAL SKIN SUBSTITUTE AND METHOD OF PREPARATION THEREOF

(71) Applicant: INDIAN INSTITUTUE OF TECHNOLOGY DELHI, New Delhi (IN)

(72) Inventors: Veena KOUL, New Delhi (IN); Arani Varamuni PRASAD THANUSHA, New Delhi (IN)

(73) Assignee: INDIAN INSTITUTUE OF TECHNOLOGY DELHI, New Delhi (IN)

(21) Appl. No.: 17/764,523

(22) PCT Filed: Sep. 25, 2020
(86) PCT No.: PCT/IN2020/050821

§ 371 Date: Mar. 28, 2022

(30) Foreign Application Priority Data
Sep. 26, 2019 (IN) 201911038914

(51) Int. Cl. A61L 27/60; A61L 27/56; A61L 27/22; A61L 27/20; A61L 27/54; A61F 2/10

(52) U.S. Cl. CPC A61L 27/60; A61L 27/56; A61L 27/222; A61L 27/20; A61L 27/54; A61F 2/105; A61L 2430/40

(57) ABSTRACT

The present invention relates to a novel acellular artificial skin substitute or scaffolds comprising biopolymer and bioactive components and the process of preparing said artificial skin substitute. The novel artificial foam-based skin substitute scaffold of the present invention addresses the problems in the prior art by providing a biocompatible, biodegradable, Non-immunogenic, non-irritant and a cost-effective scaffold.

### TECHNICAL FIELD

The present invention relates to the field of artificial skin substitutes. More particularly, the present invention relates to a novel acellular artificial skin substitute or scaffolds comprising biopolymer and bioactive components.

### BACKGROUND

The mammalian skin is the outer covering of the body and the largest organ of the integumentary system which protects the vital organs of the body surface. Skin defect arises when the skin is damaged because of inflammation, ulceration, trauma, burn, tumor surgery, congenital malformations and the like. The common process of biological wound healing is succeeded through four distinct and highly programmed phases: hemostasis, inflammation, proliferation and remodeling. For effective healing, all the four phases must occur in the proper order that involves soluble mediators, blood cells, extracellular matrix, and parenchymal cells in a given time frame.

Generally, loss of signal to repair acute and chronic wounds leads to impaired healing due to failure to follow the normal stages of healing. This leads to a state of pathologic inflammation because of incomplete or uncoordinated healing process. To solve the problem, various skin substitutes are used. Skin grafts constructed from the patient's own skin (autografts), from other human donors, most commonly, cadaver skin (allografts) and from animals (Xenografts). However, use of such skin grafts pose the challenge of infection or, in the case of cadaver skin, rejection. To overcome this challenge, various artificial skin substitutes have been developed using different biocompatible components such as chitosan, collagen, gelatin, Hyaluronic Acid etc. some of which are disclosed in relevant patent and non-patent literatures) listed below:

U.S. Pat. No. 5,686,091 discloses a structurally rigid biodegradable foam scaffold useful for cell transplantation. The foam can be loaded with nutrients and/or drugs that are eluted from the foam during transplant to promote growth of the cells. The foam scaffold of U.S. Pat. No. 5,686,091 contains polylactic acid (PLLA) and naphthalene.

U.S. Pat. No. 6,306,424 relates to porous, biocompatible and bioabsorbable foams that have a gradient in composition and/or microstructure that serve as a template for tissue regeneration, repair or augmentation. These foams can be made from blends of absorbable and biocompatible polymers such as polymerized ε-caprolactone (PCL), polymerized glycolide (PGA) or polymerized (L) lactide (PLA).

Hydrogel platforms composed of biopolymer gelatin, and glycosaminoglycan's (Hyaluronic acid and Chondroitin sulfate) incorporated with Asiatic acid (a triterpenoid) and nanoparticles (Zinc oxide and Copper oxide) have also been proposed for second degree burn wounds in Materials Science and Engineering: C, 1 Aug. 2018, Volume 89, Pages 378-386.

RSC Advances, 2018, 8, 16420 proposes nanofibrous acellular artificial skin substitute composed of mPEG-PCL grafted gelatin/hyaluronan/chondroitin sulfate/sericin for second degree burn care.

Biomaterials, 2016, 88, 83-96 proposes co-cultivation of keratinocyte-human mesenchymal stem cell (hMSC) on sericin loaded electrospun nanofibrous composite scaffold (cationic gelatin/hyaluronan/chondroitin sulfate).

A material that can be applied immediately after burn excision to “temporize” the wound bed, becomes integrated as a “neodermis,” resists contraction and infection, and provides the grounding for the second stage (an autologous, cultured composite skin) has also been disclosed in the Journal of burn care & research: official publication of the American Burn Association, January 2012; 33(1): 163-73 Journal of Biomedical Material Research, 1981 January; 15(1): 9-18 discloses a newly developed gelatin-based spray-on foam bandage for use on skin wounds. The aqueous foam is sprayed from aerosol containers and effectively covers and washes uneven wound surfaces. The foam dries to form an adherent and stable three-dimensional matrix which diminishes evaporative water losses. The foam possesses antimicrobial activity against gram-positive, gram-negative, and fungal contaminants. Surfactant and stabilizers are used to prepare sprayable foam scaffold.

Journal of Biomaterials Science Polymer Edition, February 2007; 18(12): 1527-45 discloses a highly porous collagen-based biodegradable scaffold as an alternative to synthetic, non-degradable corneal implants. The developed method involved lyophilization and subsequent stabilization through N-ethyl-N′-[3-dimethylaminopropyl] carbodiimide/N-hydroxy succinimide (EDC/NHS) cross-linking to yield longer lasting, porous scaffolds with a thickness similar to that of native cornea (500 micron). For collagen-based scaffolds, cross-linking is essential. However, it has direct effects on physical characteristics crucial for optimum cell behavior.

Several acellular temporary skin substitutes like BIOBRANE®, INTEGRA®, ALLODERM™, OASIS Wound Matrix™ etc. are known in the prior art. However, all of these suffer several disadvantages such as risk of infection, reduced or limited vascularization, poor mechanical integrity, immune rejection and very high cost.

It is important to note that all the artificial skin substitutes including the acellular skin substitutes disclosed above are either not easily available at affordable costs or are hypersensitive and tedious, thus, not readily acceptable by the receiver. Hence, there is a need for an improved acellular artificial skin substitute which is not hypersensitive, is cost-effective, readily available and addresses the above problems in the art.

The present invention overcomes the aforesaid drawbacks and provides an improved acellular artificial skin substitute comprising biopolymer and bioactive components, for effective healing of various wounds, such as burn wounds and for trauma care. The acellular artificial skin substitute of the present invention is a biodegradable and biocompatible foam-based scaffold. The acellular artificial skin substitute of the present invention mimics the native extracellular matrix (ECM) properties and exhibits excellent adhesive and wound healing properties. The process of preparation and various applications of the said acellular artificial skin substitute have also been disclosed.

### OBJECTIVE OF THE INVENTION

An important objective of the present invention is to provide a highly effective and improved acellular artificial skin substitute.

Another important objective of the present invention is to provide an acellular artificial skin substitute comprising biopolymer and bioactive components in specific proportions.

Yet another objective of the present invention is to provide an acellular artificial skin substitute as a biodegradable and biocompatible foam-based scaffold.

Another objective is to provide an acellular artificial skin scaffold which can be sterilized by gamma sterilization.

Yet another objective is to provide a process for preparation of the acellular artificial skin substitute.

Another objective is to provide an acellular artificial skin substitute for application in skin grafts and healing of various wounds such as burns and for trauma care.

Yet another objective is to provide a process for preparing the acellular artificial skin substitute by selective crosslinking concentration to provide an optimized biodegradation time point.

Still another objective is to provide an acellular artificial skin substitute with an improved release profile and better mechanical properties.

Still another objective is to provide a wound dressing material for use on different types of chronic wounds, such as diabetic, ulcer and burn wounds.

### BRIEF DESCRIPTION OF THE FIGURES

The above and other features, aspects, and advantages of the subject matter will be better understood with regard to the following description and accompanying drawings where:

FIG. 1: Representative 3-D structure of the acellular foam-based artificial skin scaffold

FIG. 2: Schematic representation of the acellular foam-based artificial skin scaffold, wherein G—Gelatin, H—Hyaluronic acid (HA), C—Chondroitin sulphate, CS—Cross-linker

FIG. 3: Artificial skin scaffold with porous upper layer and non-porous bottom layer (a) Cross-linked porous upper layer (b) Non-cross-linked non-porous bottom layer.

FIG. 4: Scanning Electron microscopy analysis: (a-b) Scanning electron microscopy images of sterilized porous upper layer at 60&150×, (c-d) Surface view of sterilized non-porous bottom layer, 60× & 150×

FIG. 5: Bar graphs showing degradation studies in unsterilized and sterilized acellular artificial skin scaffold (a) Bar graph showing gelatin degradation using Bradford Assay (b) Bar graph showing release profile of glycosaminoglycans (CS+HA) using dimethyl methylene blue (DMMB) assay

FIG. 6: Attenuated Total Reflection Fourier Transform Infrared Analysis (ATR-FTIR) of scaffolds unsterilized and sterilized (2.5 Mrd) scaffold

FIG. 7: Thermal analysis of the foam-based scaffolds

(a) Differential Scanning calorimeter (DSC) thermogram for unsterilized scaffold (first heating scans)
(b) DSC thermogram for unsterilized scaffold (first cooling and second heating scans)
(c) DSC thermogram for sterilized scaffold (first heating scans)
(d) DSC thermogram for sterilized scaffold (first cooling and second heating scan
(e) Thermogravimetric analysis (TGA) of unsterilized foam-based scaffold
(f) TGA of sterilized foam-based scaffolds.

FIG. 8: shows cellular proliferation by Lactate dehydrogenase (LDH) assay after 24 and 48 hours.

FIG. 9: Biocompatibility studies

(a) Biocompatibility studies carried out in test and reference animals using the acellular artificial skin scaffold: Histopathology analysis of vital organs of test and reference animals on day 7, 14, 21 and 28 (10× magnification).
(b) Biocompatibility studies carried out in test and reference animals using the acellular artificial skin scaffold: Histopathology analysis of Lymph node of test and reference animals on day 7, 14, 21 and 28 (10× magnification).
(c) Biocompatibility studies carried out in test and reference animals using the acellular artificial skin scaffold (c) Histopathology analysis of tissue response around the artificial skin scaffold in test and reference animals on day 7, 14, 21 and 28 (10× magnification).

FIG. 10: In-vivo wound healing experiment
(a) Representative photographs for second degree burn wound at day 0, 7, 14, 21 and 28
(b) Wound contraction assay of wound tissue in foam base and sham group at day 7, 14, 21 and 28. Data are represented as mean±SD; n=6 rats
(c) Hematoxylin and Eosin (H&E) stained histological section of wound tissue at day 28 (10× magnification).
(d) Score card of Haematoxylin and Eosin (H&E) stained histological section of wound tissue at day 7, 14, 21 and 28 days.
(e) Quantification of Tumor Necrosis Factor (TNF)-α day 0, 7, 14 and 21.
(f) IL-1α level healthy, sham, treated and reference groups on day 0, 7, 14, 21, 28
(g) Quantification of C3a levels in rat serum healthy, sham, treated and reference groups on day 0, 7, 14, 21 and 28.

### SUMMARY OF THE INVENTION

The present invention provides an acellular artificial skin substitute comprising:
a) a cross-linked porous upper layer, and
b) a non-cross-linked non-porous bottom layer
   characterized in that the said acellular foam based artificial skin substitute comprises a biopolymer and a bioactive component in specific proportions.

The present invention further provides an acellular artificial skin substitute which is a foam-based scaffold and comprises a biopolymer and bioactive components.

A process of preparing said artificial skin substitute is also provided along with its applications in skin grafts and wound dressings.

### DETAILED DESCRIPTION

The details of one or more embodiments of the invention are set forth in the accompanying description below including specific details of the best mode contemplated by the inventors for carrying out the invention. The embodiments of the invention which are apparent to a person skilled in the art after reading the present disclosure and on applying the common general knowledge of the technical field are within the scope of this invention.

### Definitions

The use of “comprise”, “comprises”, “comprising”, “contain”, “contains”, “containing”, “include”, “includes”, and “including” are not intended to be limiting. It is to be understood that both the foregoing general description and this detailed description are exemplary and explanatory only and are not restrictive.

Unless otherwise defined, scientific and technical terms used herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art.

The term “acellular” means the artificial skin substitute or scaffold does not contain any cells embedded in the scaffold. The term “scaffold” further means the materials that have been engineered to cause desirable interactions to contribute to the formation of new functional tissues for medical purposes. The terms “acellular skin substitute” and “scaffold” have been interchangeably used in the present invention.

In context of the present invention, the term “Sham” means untreated, term “control/reference” means the standard marketed product and the term “test/treated” means the invented scaffold.

Accordingly, the present invention provides an acellular artificial skin substitute having a bi-layered structure, characterized in that the said skin substitute comprises of biopolymer and bioactive components.

In a principle embodiment, the present invention provides an acellular foam based artificial skin substitute comprising:
c) a cross-linked porous upper layer, and
d) a non-cross-linked non-porous bottom layer
characterized in that the said acellular foam based artificial skin substitute comprises a biopolymer in the range of 95 to 99% and a bioactive component in the range of 1 to 5% based on the biopolymer weight.

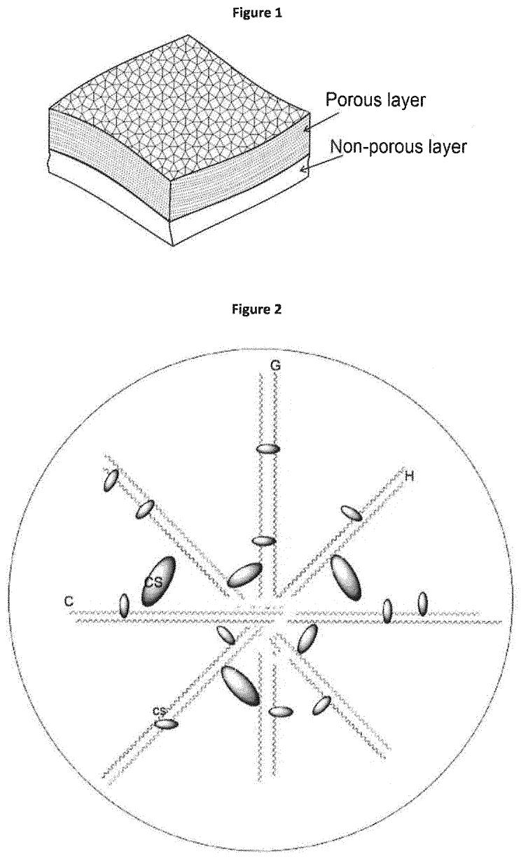

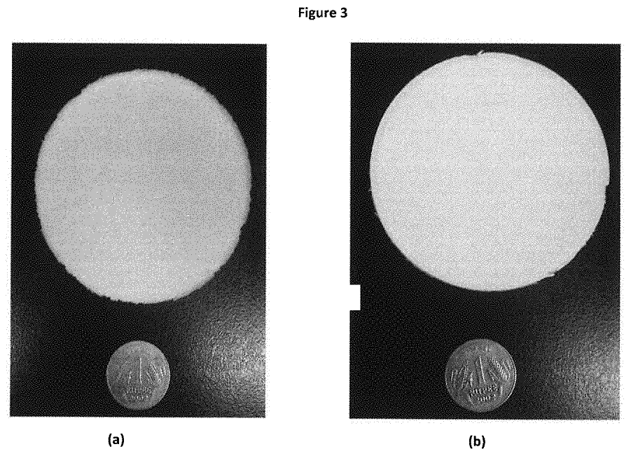

In a principle embodiment, the present invention provides an acellular foam based artificial skin substitute which is bi-layered (FIG. 1).

In an embodiment, the acellular artificial skin substitute is a scaffold of 2-4 mm proportion.

In another embodiment, the porous upper layer is crosslinked with a crosslinker selected from the group consisting of EDC, glutaraldehyde, natural polyphenolic crosslinkers like caffeic acid, genipin and tannic acid.

In yet another embodiment, the concentration of the said crosslinker ranges between 1-10 mM.

In another embodiment, the biopolymer is selected from gelatin, elastin, collagen, pectin, laminin, fibronectin and mixtures thereof.

In yet another embodiment, the bioactive component is selected from hyaluronic acid, chondroitin sulfate, Dermatan sulfate and keratin sulfate and mixtures thereof.

In yet another embodiment, the pore size of the said acellular artificial skin substitute is in the range of about 60 to about 300 μm.

In an embodiment, the artificial skin scaffold is sterilized either by gamma radiation or by ethylene oxide.

In yet another aspect of the invention, a process for the preparation of acellular foam based artificial skin substitute comprising the steps of:
i. obtaining a solubilized biopolymer by dissolving said biopolymer in water,
ii. adding bioactive components to the solubilized biopolymer and stirring continuously at appropriate temperature and for appropriate time to obtain a dissolved composite viscous solution,
iii. placing the dissolved composite viscous solution under foam maker,
iv. casting the composite viscous solution as obtained in step (iv) on a petri-plate and distributing the same homogenously for uniform thickness
v. adding the crosslinker in a specific manner to selectively crosslink the composite viscous solution such that only the upper layer gets crosslinked
vi. obtaining the selectively crosslinked acellular artificial skin substitute having a cross-linked porous upper layer, and a non-cross-linked non-porous bottom layer,
vii. removing excess cross linker by repeated washing and
viii. lyophilizing the acellular artificial skin substitute.

In another embodiment of the invention, the solubilized biopolymer is obtained by dissolving in water at 40-45 for 10-30 minutes.

In yet another embodiment, the bioactive components are added to the solubilized bio polymer obtained in step (i) of the process is stirred continuously at 40-45° C. for 1-4 hours to obtain a dissolved composite viscous solution.

In another embodiment, the dissolved composite viscous solution of step (ii) of the process is put under foam maker at 1200 to 15,000 rpm for 1-5 minutes.

In yet another embodiment, the viscous solution obtained in step (iii) of claim 1 is cast on the petri plate by homogeneous distribution and keeping the petri plate undisturbed for 30 minutes at 25-30° C.

In another embodiment, the homogenously cast viscous solution is selectively cross-linked by adding the crosslinker in a specific manner for 10-20 minutes at 4° C. to 8° C. to obtain the acellular skin substitute having a cross-linked porous upper layer, and a non-cross-linked non-porous bottom layer with uniform thickness. The artificial skin substitute thus prepared is kept for pre-freezing at −40 to −30° C. for 2-3 hours followed by lyophilization in the following manner:
Step 1: at −30° C. for 1-3 hours
Step 2: at −20° C. for 2-4 hours
Step 3: at −10° C. for 2-3 hours
Step 4: at 1° C. for 3-4 hours
Step 5: at 5° C. for 2-3 hours
Step 6: at 10° C. for 2-3 hours
Step 7: at 20° C. for 3-4 hours
   of the same vacuum of 0.03-0.06 Mbar pressure in all the lyophilization steps. (xi) After lyophilization for 15-20 hours, the developed artificial skin substitute is ready for application.

In an embodiment, the acellular artificial skin substitute is used as a skin graft.

In another embodiment, the acellular artificial skin substitute is used as a wound dressing in healing of wounds, preferably the wounds associated with burn and trauma care.

In another embodiment, a kit comprising of the acellular foam based artificial skin substitute of the present invention along with instructions for its use.

In yet another embodiment, a method for treating the wound related infections comprising applying the wound dressing comprising the artificial skin substitute to the affected area/part of a subject in need of such treatment.

### Synthesis and Evaluation of the Acellular Skin Substitute:

Optimized concentrations of biopolymers and the bioactive components were weighed and dissolved in distilled water. After dissolution of biopolymer and bioactive components were admixed into the biopolymer solution and stirred for appropriate time. The complete solution was later kept under foam maker stage and the foam morphology was developed. Crosslinking solution of different concentrations were prepared separately for the desired degradation and added to the aforesaid solution. The crosslinked solution was directly cast on a petri plate and air dried to obtain acellular skin substitute. Skin substitutes thus obtained were washed thrice with distilled water and lyophilized for further studies.

The lyophilized samples were gamma sterilized at 2.5 Mrd and characterized for pre and post sterilization for assessing the properties of sterilized and unsterilized acellular artificial skin substitute by the following studies:
Surface morphology analysis—SEM
Degradation studies—Bradford assay
Release studies of HA+CS—DMMB assay
Functional group analysis by ATR-FTIR
Thermal stability by DSC
Mechanical studies by Tensile strength

The present invention uses biopolymer such as gelatin and glycosaminoglycans like hyaluronic acid and chondroitin sulfate for the fabrication of acellular artificial skin substitute, which is biocompatible and non-immunogenic The acellular artificial skin substitute of the present invention substantially mimics the native ECM properties and exhibits excellent adhesive and wound healing properties.

The bioactive component used in the present invention is selected from the group comprising of hyaluronic acid, chondroitin sulfate, Dermatan sulfate and keratin sulfate. The role of bioactive component is in recruitment of fibroblasts thereby increasing the activity of native cells to regenerate the wound tissue in early days. The bioactive component helps in regulating tissue injury in wound healing and also monitors several aspects of tissue repair, including activation of inflammatory cells to enhance immune response and provides structural framework to the fibroblasts and epithelial regeneration.

The acellular artificial skin substitute of the present application helps in mimicking the native ECM, helps in cellular attachment, possess Arginyl-glycyl-aspartic acid (RGD) sequence in their structure and non-immunogenic to the host. The biodegradable nature of the acellular artificial skin scaffold helps in the healing of wounds without any adverse effects.

The fabricated acellular artificial skin substitute is of uniform thickness in the range of 2-4 mm comprising upper layer and bottom layer.

The acellular artificial skin substitute is made up of mainly gelatin which is non-immunogenic to the body and is cost effective.

The acellular artificial skin substitute is selectively cross-linked so that rate of biodegradation of scaffold is equivalent to the rate of healing of the skin.

### EXAMPLES

The following examples are given by way of illustration of the present invention and should not be construed to limit the scope of present disclosure. It is to be understood that both the foregoing general description and the following detailed description are exemplary and explanatory only and are intended to provide further explanation of the subject matter.

### Materials:

Gelatin (Bloom No. 250) from Nitta Gelatin India Limited, Sodium hyaluronate from Kikoman Biochemica company, Chondroitin sulfate from Yantai Dongcheng Biochemicals co. LTD. All the above components are certified with pharmaceutical grade. EDC from Spectrochem, Bradford reagent and DMMB procured from Sigma-Aldrich, L929 cells were received from NCCS Pune, Penicillin-streptomycin solution, fetal bovine serum (FBS) and Dulbecco's modified Eagle's medium (DMEM) were obtained from Gibco Life Technologies (NY, USA).

### Synthesis and Evaluation of Foam-Based Scaffold (Artificial Skin Substitute):

97.75% (w/v) of biopolymer (gelatin) and 2.25% of bioactive components (HA and CS) and 10 mM EDC was used to fabricate the scaffolds. The gelatin was dissolved in distilled water and kept on heating magnetic system stirrer for 20 minutes at 45° C. HA and CS were separately dissolved in 1 ml. distilled water.

After gelatin gets completely solubilized, HA and CS solutions were added to the solubilized gelatin solution and kept for stirring for 2 hours at 45° C. The dissolved composite viscous solution was put under foam maker at 1200 to 15,000 rpm for 1-5 minutes.

The prepared viscous solution was poured on to a 90 mm sterilized petri plate for homogenous spreading without disturbing the plate at 25-30° C. for 30 minutes.

EDC solution (crosslinker) was added in a specific manner with respect to time, temperature, concentration and amount so that only upper layer gets selectively crosslinked and the bottom layer remain non-crosslinked. The procedure for EDC crosslinking was carried out at 4° C. for 10 to 20 minutes.

After 10 to 20 min, the EDC was removed completely by repeated washing with distilled water. The artificial skin substitute was kept for pre-freezing at −40 to −30° C. for 2-3 hours; followed by lyophilization for 15 to 20 hours in the following manner:
Step 1: at −30° C. for 1-3 hours
Step 2: at −20° C. for 2-4 hours
Step 3: at −10° C. for 2-3 hours
Step 4: at 1° C. for 3-4 hours
Step 5: at 5° C. for 2-3 hours
Step 6: at 10° C. for 2-3 hours
Step 7: at 20° C. for 3-4 hours

A vacuum of 0.06 Mbar pressure was maintained in all the lyophilization steps.

### Surface Morphology Analysis:

The morphology of the developed foam scaffold was analyzed using Scanning Electron Microscope, (ZEISS Model EVO 50). Cross sectional images of sterilized and unsterilized scaffolds were performed to evaluate the change in the morphology.

### Results:

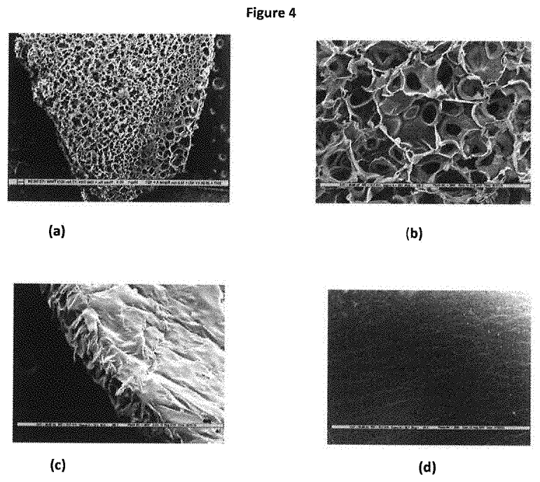

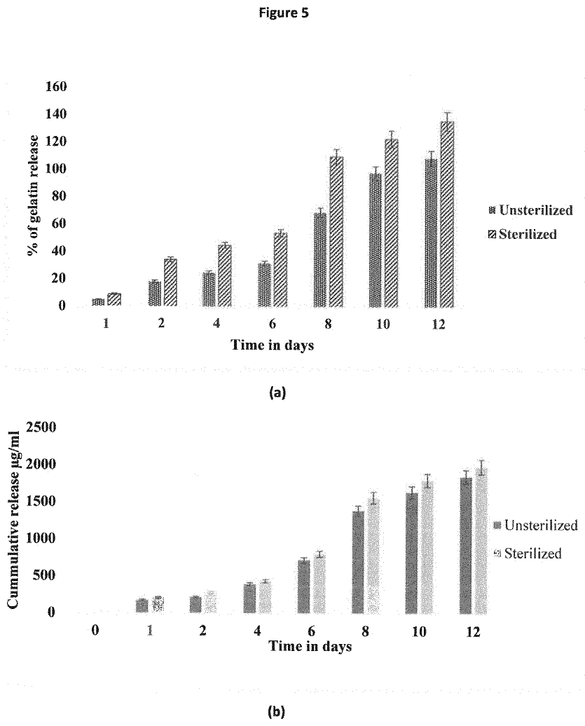

The developed foam-based scaffold is characterized by scanning electron microscopy to visualize surface and cross-sectional morphologies (FIG. 4). The foam-based scaffolds displayed a highly porous, interconnected structure (FIG. 4). The morphology of bottom layer represents smooth surface. (FIG. 4 c and d), whereas pores present in upper layers have honeycomb structure with both micro and macros porous morphology (FIG. 4 a and b). The pore size was found to be in the range of 60-300 μm. This interconnected porous structure is expected to have good permeability for nutrients and to support cellular growth.

### Degradation Studies:

The degradation profile of sterilized and unsterilized foam scaffolds was carried out by Bradford assay (FIG. 5 a). The release of gelatin from the foam composite was studied by placing the scaffold in 1 ml phosphate buffer with 1% sodium azide (pH 7.4) in an incubator shaker running at 50 rpm and 37±0.2° C. 500 μl of supernatant was collected until the scaffold got degraded and fresh media of equal volume was refilled.

### Results:

From the degradation studies it was observed that the scaffolds which were gamma sterilized degraded faster than that of the unsterilized scaffolds. The reason could be the gamma radiation may form chain scissions and lead the polymeric structure to degrade faster. The degradation is higher in 2.5 Mrd dose compared that of unsterilized scaffold. On first day, the unsterilized scaffold released gelatin of 64% whereas 2.5 Mrd dose released 78% of gelatin in to the medium in invitro conditions. On 12th day the same scaffolds released the complete gelatin in both unsterilized and sterilized scaffolds respectively (FIG. 5a).

### Release Profile of Glycosaminoglycans (GAGs)

To evaluate the release profile of glycosaminoglycans (CS+HA) from the sterilized and unsterilized scaffolds, the dimethyl methylene blue (DMMB) assay was used. The scaffolds of ˜10 mm diameter was immersed in 1 ml phosphate buffer were transferred to incubator shaker at 50 rpm at 37+2° C. 200 μl of sample was collected after pre-determined time intervals (1 day till 12 days) and refilled with fresh media of equal volume. The degradation profile of sterilized and unsterilized foam scaffolds was carried out on Bradford assay for gelatin and dimethyl methylene blue (DMMB) assay for HA and CS. The standard curve was obtained by using known concentration of chondroitin sulfate.

### Results:

The quantification of release of glycosaminoglycans into the system in a specific time is an important factor to consider the efficacy of the scaffold in wound healing treatment. The release of glycosaminoglycans into the medium primarily depends upon the crosslinking rate. The foam-based scaffold is designed to degrade in to 10-15 days, so that the release of bioactive components into the wound in the initial days will help in faster the healing rate (FIG. 5b). The gamma radiated foam-based scaffolds released the bioactive components faster than the unsterilized scaffolds, which may be due to chain scission and breakage of intramolecular attraction of bonds in the structure of the scaffold.

### Attenuated Total Reflection Fourier Transform Infrared Analysis (ATR-FTIR):

FT-IR spectra were collected with a Thermo Nicolet 380 spectrometer equipped with ATR accessory and the spectra resolution was 4 cm-1. The spectrum of the pre and post sterilized samples were attained by placing the foam scaffold onto germanium crystal without any additional sample preparation. The spectra were the result of 36 scans.

### Results:

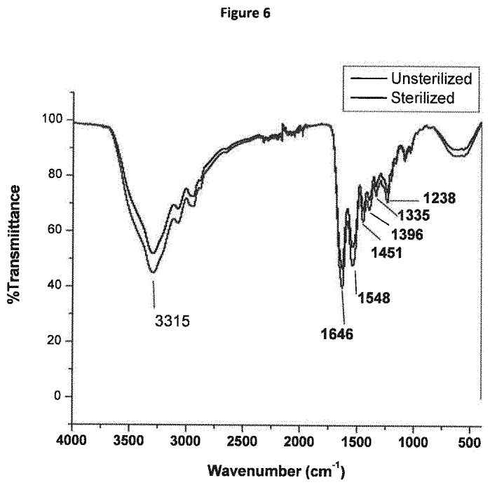

ATR-FTIR spectra of foam-based scaffolds of unsterilized and sterilized (2.5 Mrd) scaffolds are shown in FIG. 6. Both spectra show a broad band at 3315 cm⁻¹ which could be due to the presence of —OH, —NH stretching vibrations. In both the spectra, bands at 1646 and 1548 cm⁻¹ are assigned to amide I and amide II bands, respectively. A band of peaks from 1335-1451 cm⁻¹ could be assigned to C—H₂ bending vibrations. The overlay of spectra (unsterilized and sterilized) confirms that there is no change among stretching and bending vibrations.

### Differential Scanning Calorimetry

Calorimetric measurements were performed using Q2000 Differential Scanning Calorimeter (DSC) thermo gravimetric analyzer (Q-500). Waltham, USA under nitrogen atmosphere for pre and post sterilized samples. The measurements were carried out on known amounts of scaffold (3-4 mg) and the samples were hermetically sealed in aluminum pans. DSC and TGA traces were recorded in nitrogen atmosphere at a heating rate of 10° C./min and 20° C./min.

### Results:

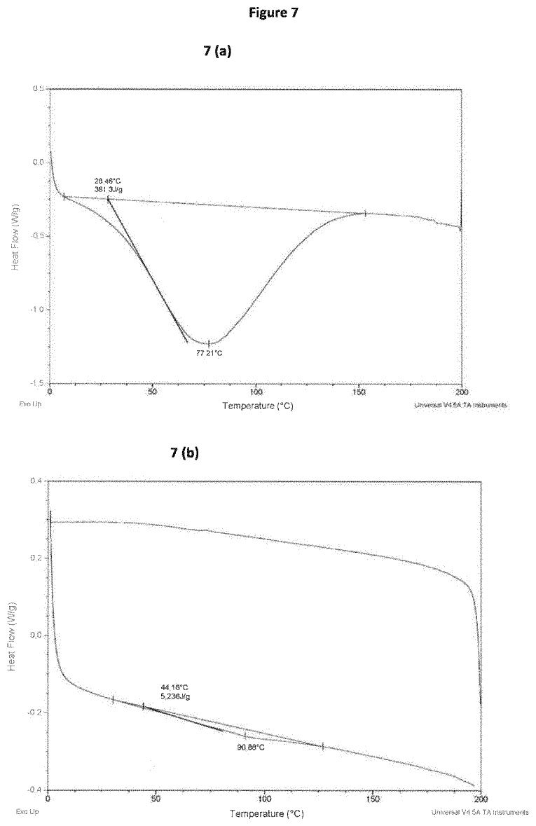

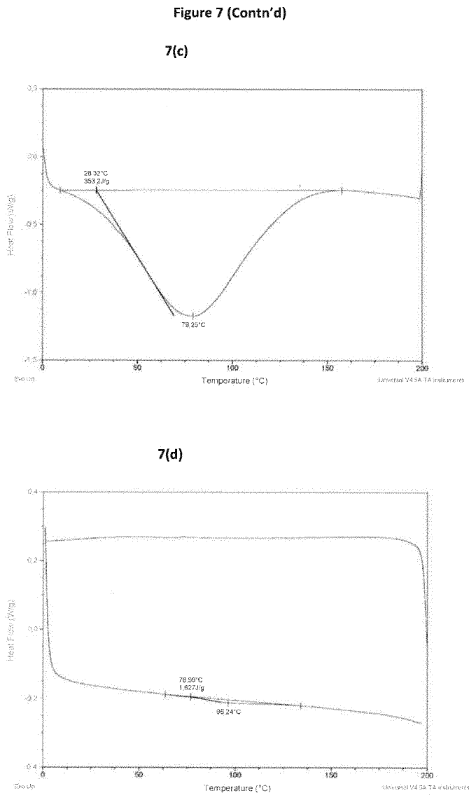

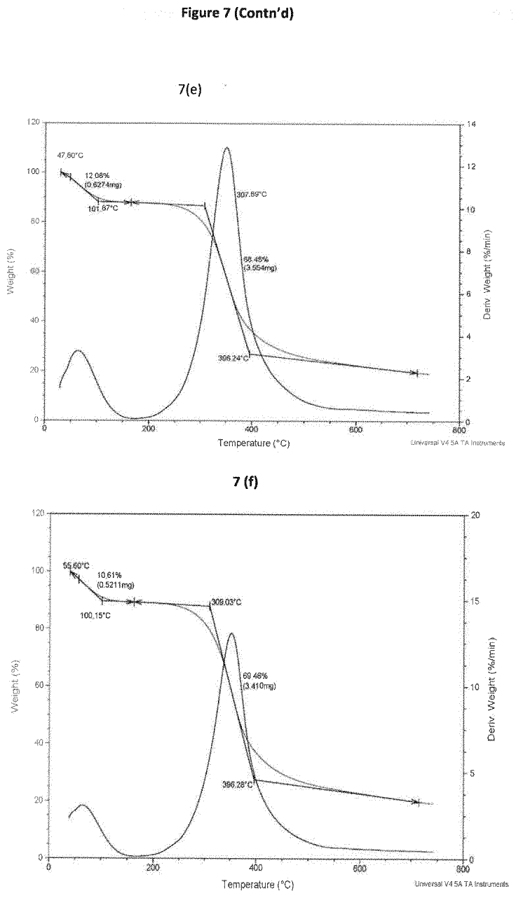

The DSC thermogram of the foam-based scaffolds in FIG. 7 (a-d) shows an endotherm peak due to the transition of helical structure of collagen. The enthalpy of denaturation associated with the endothermic peak directly depends on the structure of gelatin and hydrogen bonds whereas the exothermic peak relates to the covalent crosslinking of the scaffold. The endothermic peak at 70° C. was similar in comparison to all the scaffolds, whether sterilized or unsterilized. DSC data confirms that sterilization by gamma radiation does not affect the thermal stability of the developed foam-based scaffolds of the present invention.

### Thermal Characterization

Samples before and after sterilization were characterized using Differential Scanning Calorimetry (DSC) and Thermogravimetric analysis (TGA). DSC and TGA traces were recorded in nitrogen atmosphere at a heating rate of 10° C./min and 20° C./min using DSC 2000 and thermo gravimetric analyzer (Q-500).

In the first heating scan a broad endothermic transition at 77° C. for unsterilized and at 79° C. (FIG. 7 e & f) for sterilized scaffolds was observed in the temperature range of 0-200° C. This could be due to loss of water that could be adsorbed or bound or entrapped. This transition is irreversible as no endothermic peak is observed in second heating scan. However, in second heating scan, endothermic shift of base line was observed which could be due to glass transition temperature. The glass transition temperature was found to be at 90° C. for unsterilized and 96° C. for sterilized foam-based scaffold. In complementary, TGA traces were also recorded in temperature range 0-700° C. (FIGS. 7e and f). TGA thermogram for unsterilized and sterilized foam-based scaffolds shown similar thermal degradation pattern, as 10-12% weight loss in the temperature range 0-100° C. was observed for both unsterilized and sterilized (2.5 Mrd) foam-based scaffold (FIGS. 7e and f). The weight loss corresponds to endothermic transition observed during first heating scan. Polymer degradation was observed at 200-400° C. for both cases. These results indicate sterilization by gamma radiation doesn't affect the thermal stability of developed foam-based scaffolds.

### Tensile Properties:

The tensile test was carried out on sterilized and unsterilized foam-based scaffolds using micro-tensile tester (H5KS; armed with 1 kN load cell). The scaffolds were cut into length 60 mm, width 8 mm, thickness 3 mm and the test were conducted at a crosshead speed of 10 mm/minute (ASTM-D 638).

### Results:

An effective wound-based scaffold must possess the biocompatibility, biodegradability and mechanical property to function properly in the given environment. Tensile strength of a scaffold is an important parameter to analyze the resistance of the developed foam scaffold to understand the deformity caused due to stress. According to research reports, gamma sterilized scaffolds can lead to chain scission in the gelatin structure and ultimately leads to lower tensile strength with a faster degradation rate. In agreement with the statement, the unsterilized scaffolds showed greater tensile strength than the sterilized scaffolds of 2.5 Mrd dose (Table 1).

TABLE 1
Tensile studies of un-sterilized and sterilized scaffold

| Scaffold | Tensile strength (MPa) | Elongation at max load (%) |
|---|---|---|
| Unsterilized | 1.134 + 0.28 | 42 + 3.1 |
| Sterilized | 0.973 + 0.13 | 40.3 + 3.6 |

### Cyto-Compatibility Studies:

### Cell Seeding and Characterization of Scaffolds:

Scaffolds of ≈6 mm diameter were sterilized by 72 hrs of UV exposure on each side and incubated in PBS for 4 hrs followed by incubating in complete cell culture medium (DMEM media supplemented with 1% penicillin streptomycin and 10% FBS) for 12 hrs at 37° C. (5% CO₂ incubator). After 12 hours, the medium was removed completely and seeded with ≈10000 cells L929 Mouse fibroblast cells (NCCS, Pune) on each scaffold and incubated for 3 hours for early adhesion of cells to the scaffold. Medium was then added and incubated for 24 and 72 hours for L929 to quantify Lactate dehydrogenase (LDH) and DNA. After completion of scheduled time intervals, samples were washed with PBS and stored in −80° C. for biochemical analysis.

### Lactate Dehydrogenase (LDH) Assay:

Thermo Scientific Pierce LDH Cytotoxicity assay kit was used to estimate the percentage of viable cells in the given scaffold. After predetermined time points, cell lysis buffer was added to the culture plate at 25+2° C. for 45 min at 50 rpm. To the 50 μl cell lysate, 50 μl of LDH substrate was added and incubated for 5 min and then stop solution was mixed for the enzymatic reaction to complete and assessed for absorbance at 492 nm. The control was only cells without scaffold.

### Results

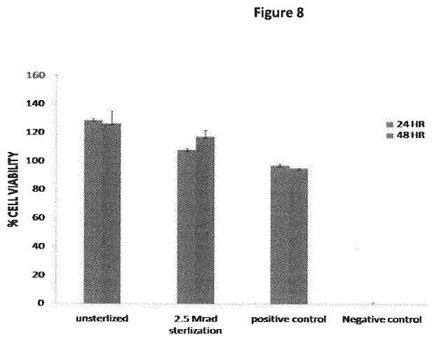

Effect of scaffolds on the cell viability of fibroblast cells was measured indirectly with the help of LDH assay (FIG. 8). The study shows that scaffolds did not exhibited any cytotoxic effect on fibroblast cell lines and promote cell proliferation in both treated and reference in comparison to control.

### Biocompatibility Studies:

Understanding the biocompatibility of the developed scaffold is an important parameter to assess before proceeding for animal study as it provides an assessment of tissue response towards host in the actual situation. To evaluate the compatibility of sterilized foam-based scaffold in biological environment on Wistar rats, blood chemistry, histopathology and inflammatory response parameters were considered.

### Methodology:

The studies were planned to examine the biocompatibility of scaffold over a time period of 7, 14, 21 and 28 days. The Wistar rats with body weight of 170+50 g and age of 8 to 12 weeks in healthy condition were procured from Central Animal Facility (AIIMS, New Delhi) under ethical approval to perform the studies (No. 40/IAEC/17).

Wistar rats were randomized into two groups (each consisted of 5 rats). Group I-Control/reference (High density polyethylene) and Group II-treated scaffold. The animals were anaesthetized using ketamine (50-80 mg/kg⁻¹) (150 μL/rat). Hair was removed at two places on the dorsal side of the body of the rats. With two small incisions, the control/reference and test/treated samples were implanted inside the cut of all experimental animals. After implantation, the incisions were sutured, and antibiotic ointment applied on the sutures. The animals were sacrificed at specific time period (7, 14, 21, and 28 days) for biochemical and histology evaluation.

### Hematological Analysis:

Histopathology analysis of implanted area tissue and vital organs was performed by sacrificing the animals by injecting an overdose of ketamine. Vital organs such as heart, lungs, liver, kidney, spleen and axillary lymph nodes and tissue of implanting area were collected and immediately fixed in 10% formalin. Samples were embedded in paraffin blocks through processing and 3 μm sections were cut using microtome. The sections were stained in hematoxylin and eosin for microscopic analysis.

At 7, 14, 21 and 28 days, 1 mL of blood was collected from the experimental animals in a test tube coated with anti-coagulant substance (EDTA). An automated complete blood count was performed using whole blood. The sample tests were analyzed for hemoglobin concentration (Hb), PCV, TLC, Polymorphonuclear cells, lymphocytes, eosinophil, monocytes, ANC, AEC, platelet count, ESR, RBC, hematocrit (Hct), mean corpuscular volume (VCM), mean corpuscular hemoglobin (HCM), mean corpuscular hemoglobin concentration (CHCM).

### Results:

Hematological analysis was done to investigate any toxicity or abnormality in blood components caused by implant scaffold. All the parameters of hematology were applied for the treated as well as control/reference samples. The values of treated samples were compared with the values of control/reference sample for the period of 7, 14, 21 and 28 days (Table 2).

It was observed that most of the values of hematology for test samples showed no statistically significant differences when compared to the values of control/reference. The number of monocytes in all the treated samples drawn at 7, 14, 21 and 28 days was high (5 to 9) as compared to that in the control/reference sample (2.3). Monocytes differentiate into macrophages and dendritic cells; hence the values could be higher due to degradation of the scaffold.

TABLE 2
Hematology analysis of control and foam-based scaffold on day 7, 14, 21 and 28

| | Control | 7 Days | 14 Days | 21 days | 28 Days |
|---|---|---|---|---|---|
| Hb | 13.17 ± 1.08 | 12.4 ± 1.01 | 12.46 ± 0.73 | 13.4 ± 0.24 | 13.76 ± 0.32 |
| Polymorph | 20.33 ± 1.24 | 24 ± 2.94 | 28.33 ± 2.62 | 26 ± 1.41 | 17.33 ± 1.69 |
| Lymph | 78.67 ± 3.85 | 66.67 ± 4.1 | 67.6 ± 3.68 | 69.66 ± 4.64 | 74.33 ± 10.20 |
| Eosino | 2 ± 0.81 | 2 ± 0 | 1.33 ± 0.47 | 2.33 ± 0.47 | 2 ± 0.81 |
| Monocytes | 2.33 ± 0.47 | 9 ± 1.41 | 5 ± 1.41 | 5.33 ± 2.86 | 9.66 ± 7.36 |
| Mchc | 32.82 ± 0.05 | 31.75 ± 0.56 | 32.53 ± 0.36 | 32.04 ± 0.51 | 33.07 ± 0.86 |
| Rdw | 15.8 ± 0.82 | 16.73 ± 1.04 | 16.6 ± 0.99 | 14.56 ± 0.61 | 15.4 ± 0.94 |

### Biochemical Analysis:

At the time of sacrifice, 2 mL of blood was collected in a test tube without anticoagulant. The test tube was centrifuged at 1000 rpm for 10 minutes to obtain the serum. An automated liver function test including (bilirubin, total proteins, albumin, Globulin, A:G ratio, Alkaline Phosphatase, SGOT, SGPT) and kidney function test including (urea, urea nitrogen, creatinine, uric acid, calcium, phosphate, sodium, potassium, chloride) were performed using serum.

### Liver Function Test:

Serum biochemistry was done to assess the renal or hepatic toxicity caused by implanted gelatin scaffold. All the parameters of LFT (Table 3) were conducted for test and control samples.

### Results:

As observed, in liver function test, the total protein, globulin, and albumin:globulin ratio, SGOT, SGPT and alkaline phosphatase did not show any statistically significant differences when compared to the values of control.

TABLE 3
Liver function test of control/reference and treated on day 7, 14, 21 and 28:

| | Control | 7 Days | 14 Days | 21 Days | 28 Days |
|---|---|---|---|---|---|
| BILI Tot | 0.04 ± 0.02 | 0.04 ± 0.03 | 0.04 ± 0.00 | 0.02 ± 0.00 | 0.02 ± 0 |
| BILI Dire | 0.02 ± 0.00 | 0.02 ± 0.00 | 0.02 ± 0 | 0.02 ± 0.00 | 0.01 ± 0.00 |
| BILI Indir | 0.02 ± 0.00 | 0.03 ± 0.02 | 0.02 ± 0.00 | 0.02 ± 0.01 | 0.02 ± 0.02 |
| Total | 7.29 ± 0.06 | 7.00 ± 0.13 | 6.82 ± 0.44 | 6.88 ± 0.08 | 6.98 ± 0.33 |
| Proteins | | | | | |
| Albumin | 4.67 ± 0.33 | 4.53 ± 0.50 | 4.48 ± 0.51 | 4.38 ± 0.27 | 4.46 ± 0.06 |
| Globulin | 2.74 ± 0.15 | 2.68 ± 0.06 | 2.89 ± 0.26 | 2.43 ± 0.26 | 2.49 ± 0.26 |
| A: G Ratio | 1.74 ± 0.24 | 1.56 ± 0.05 | 1.44 ± 0.18 | 1.72 ± 0.44 | 1.76 ± 0.19 |
| Alk Phos | 216.98 ± 11.68 | 196.82 ± 49.58 | 313.98 ± 121.98 | 268.14 ± 50.34 | 286.36 ± 28.23 |
| SGOT | 156.92 ± 6.97 | 143.97 ± 16.76 | 141.59 ± 4.49 | 145.19 ± 8.64 | 144.86 ± 1.25 |
| SGPT | 67.39 ± 0.60 | 50.79 ± 19.30 | 57.11 ± 3.67 | 56.72 ± 8.57 | 56.46 ± 5.49 |
| GGTP | 0.033 ± 0.04 | 0 ± 0 | 1.92 ± 1.53 | 0 ± 0 | 0.06 ± 0.04 |

### Kidney Function Test:

Serum biochemistry was done to assess the renal or hepatic toxicity caused by implanted gelatin scaffold. All the parameters of KFT (Table 4) were conducted for test and control samples.

### Results:

In kidney function test, no significant changes were observed in the level of urea nitrogen, creatinine, potassium etc. as compared to the control/reference. Hence, kidney did not show any abnormalities as compared to healthy animals.

TABLE 4
Kidney function test of control/reference and treated on day 7, 14, 21 and 28

| | Control | 7 Days | 14 Days | 21 Days | 28 Days |
|---|---|---|---|---|---|
| Urea | 44.46 ± 4.36 | 43.94 ± 3.09 | 47.00 ± 5.34 | 48.41 ± 4.60 | 46.49 ± 2.47 |
| Urea Nitrogen | 20.77 ± 2.03 | 20.36 ± 1.48 | 21.85 ± 2.64 | 21.73 ± 1.01 | 21.64 ± 1.23 |
| Creatinine | 0.32 ± 0.01 | 0.33 ± 0.03 | 0.34 ± 0.00 | 0.33 ± 0.01 | 0.33 ± 0.00 |
| Uric Acid | 1.02 ± 0.10 | 1.29 ± 0.10 | 1.33 ± 0.12 | 1.07 ± 0.21 | 0.98 ± 0.19 |
| Calcium | 10.3 ± 0.16 | 10.11 ± 0.18 | 10.25 ± 0.16 | 10.31 ± 0.52 | 9.77 ± 0.24 |
| Phosphate | 4.33 ± 0.77 | 5.88 ± 1.06 | 6.13 ± 0.55 | 5.11 ± 0.93 | 4.67 ± 1.31 |
| Sodium | 139.77 ± 1.41 | 139.69 ± 2.63 | 138.96 ± 2.67 | 140.13 ± 1.01 | 139.73 ± 1.69 |
| Potassium | 4.68 ± 0.32 | 4.7 ± 0.61 | 5.22 ± 1.50 | 4.9 ± 0.44 | 4.49 ± 0.18 |
| Chloride | 98.42 ± 1.08 | 99.82 ± 0.73 | 97.65 ± 2.30 | 101.51 ± 1.38 | 101.29 ± 1.11 |

### Histopathology Assay:

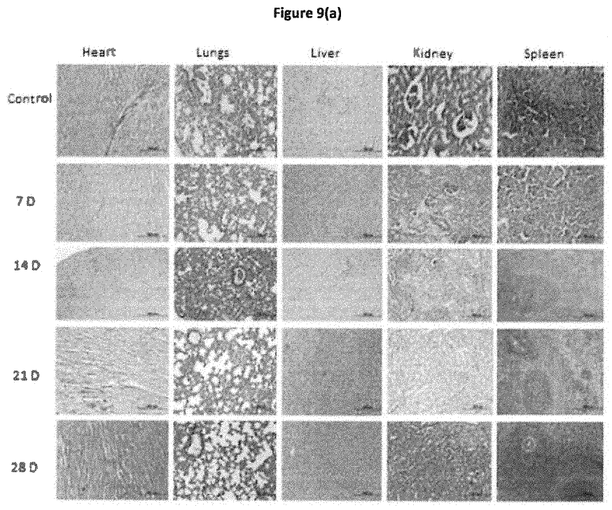

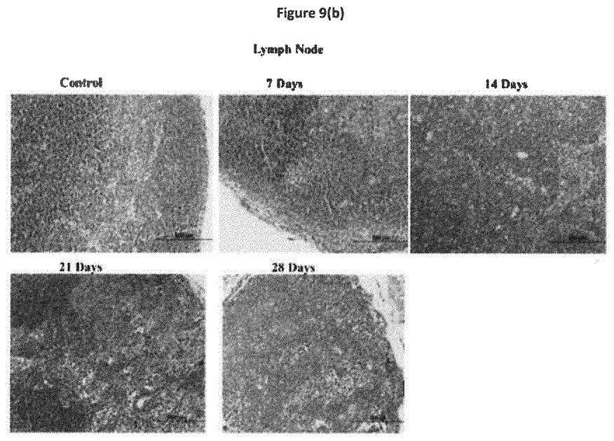

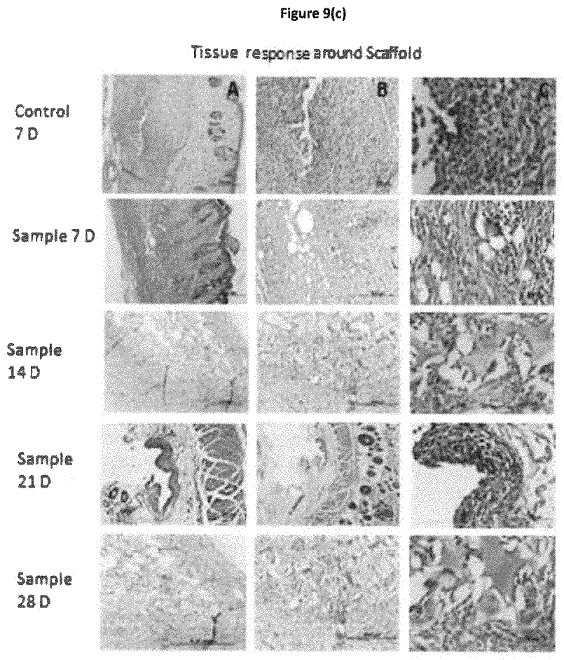

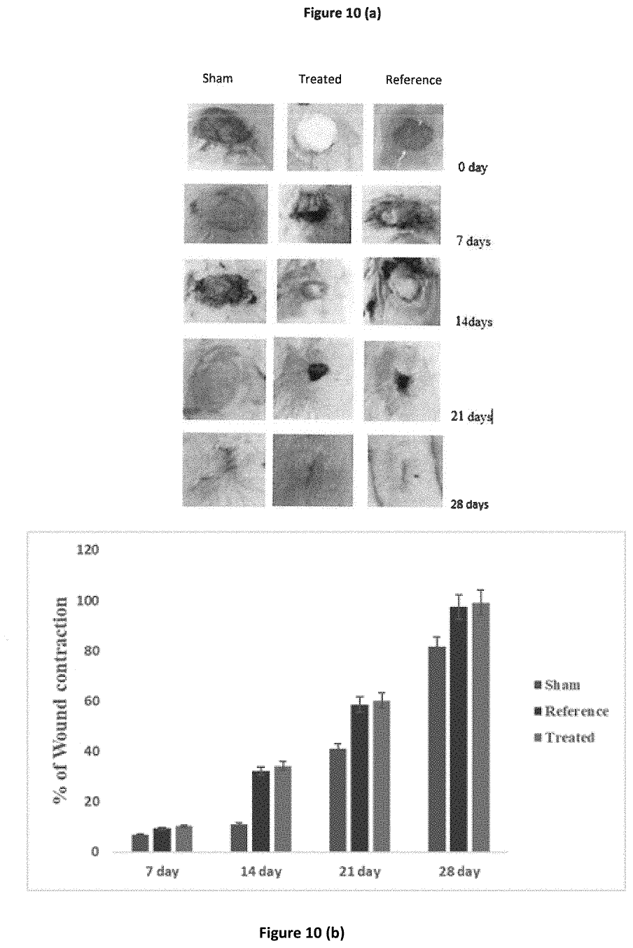

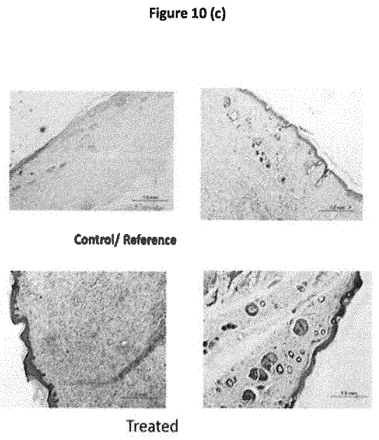

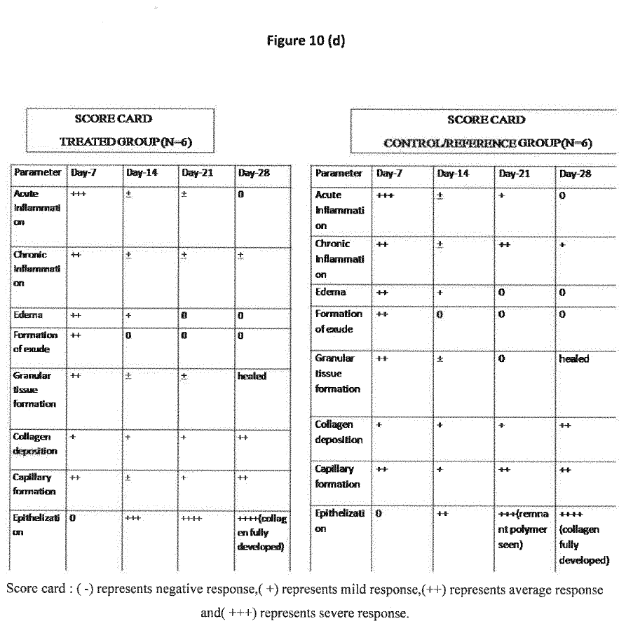

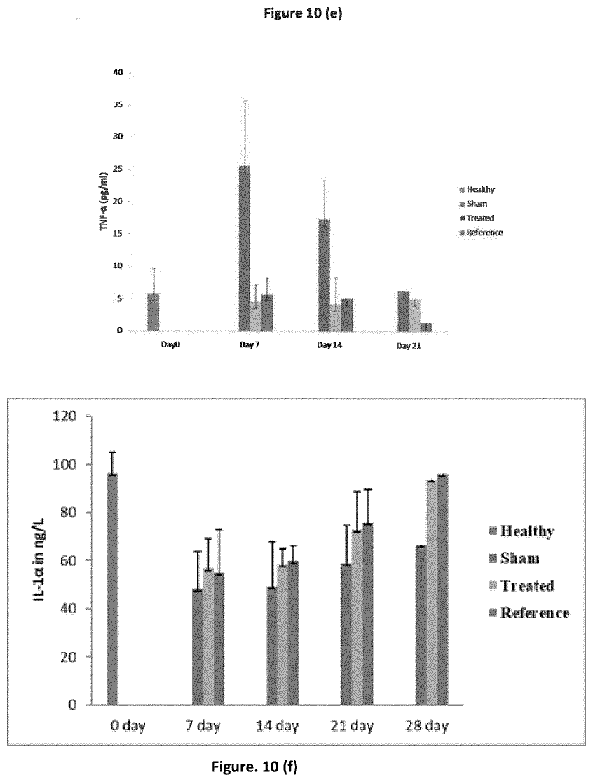

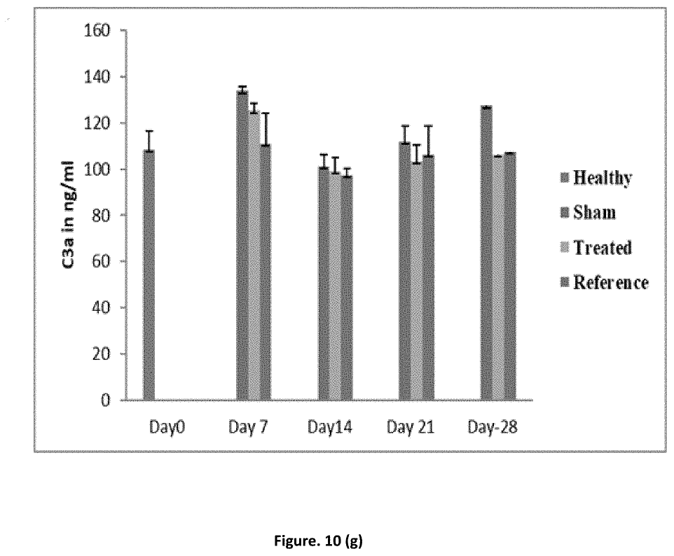

The histological analysis was carried out to investigate the inflammatory response, fibrosis, and granuloma caused by in vivo implanted scaffold. The vital organs such as heart, lungs, liver, kidney, spleen and lymph node were histologically evaluated for any abnormality after 7, 14, 21, and 28 days of implantation. There was no abnormality seen and fibrous capsule around the implants and the morphology features of the organs in all the group of test animals were similar and comparable to control (FIG. 9 a, b and c). However, the fibrous capsule formation is a well-established reaction to any implanted biomaterials and recognized as foreign body reaction. The tissue around the scaffold showed inflammatory cells and no granuloma was seen. Mild inflammation can be expected for any foreign materials or biomaterials as it could be recognized as part of the body's foreign body response

### In-Vivo Wound Healing Experiment:

The developed and sterilized foam-based artificial skin substitute or scaffold was evaluated for its efficacy in healing of second degree burn wounds in experimental male Wistar rats of weight 200-250 grams with the recommendation of animal Ethical Committee clearance of All India Institute of Medical Sciences, New Delhi (40/IAEC/17). Initially, the animals were anaesthetized using ketamine hydrochloride.

Each rat of the reference group treated group/sham group and reference groups were placed in separate cages and provided the food and water ad libitum. Second degree burn wound was inflicted upon the dorsal region of the rat. A cylindrical aluminum bar (20 mm dia) heated up to 120° C. was placed on the shaved area of skin for 20 sec to create a second degree burn wound. After 24 hrs the burnt epidermis portion was removed and applied saline immersed foam scaffold on to the wound surface and wrapped with breathable film for constant aeration to the wounds. On Day 7, 14, 21 and 28, blood was drawn for biochemical analysis, pro-healing and inflammatory markers and skin samples were removed from each rat and the area of wound was measured for wound contraction assay (FIG. 10 a). Skin from the burn wound area was taken for histopathological examination and animals were sacrificed and their organs were taken (liver, kidney, spleen, heart and lung) for histopathological examination. Quantification of pro-healing markers like hexosamine and hydroxyl proline and pro inflammatory markers like inflammatory markers like TNF-α, IL-1α, C3a through enzyme linked immunosorbent assay.

### Results:

### Wound Contraction Assay:

On day 0, the wound area was 20 mm diameter in all the groups. After applying the scaffold with treated group and integra in reference group % wound contraction of the burned area start increasing with time. It was observed that percentage of wound contraction in treated group was 10.25±2.98, 34.22±2.19, 60.11±3.20 and 99.21±2.41 on day 7, 14, 21 and 28 days respectively. The wound contraction was found to be significantly higher in treated group as compared to sham group till day 28 and slightly higher to reference group. The percentage of wound contraction in sham group was 6.93±2.93, 10.93±3.62, 40.95±1.87, 81.52±1.54 on day 7, 14, 21 and 28 days respectively. The percentage of wound contraction in reference group was 9.35±2.89, 32.3±4.42, 58.61±3.41 and 97.43±2.25 on day 7, 14, 21 and 28 days, respectively (FIG. 10b).

The in vivo 2^{nd} degree burn wound studies on Wistar rats demonstrated healing of the burn wound by histopathology and biochemical parameters. From the histopathological analysis of H&E stain (FIG. 10c) it was observed epithelization was complete on 28^{th} day for treated (skin substitute) as well as reference scaffolds. The score card of the histopatholgy pictures of H&E stain, for artificial skin scaffold was compared to reference scaffold for 7, 14, 21 and 28 days, it provide an insight on acute and chronic inflammatory parameters and suggested no scar formation, enhanced collagen synthesis and epithelization (Score card of histopathology, (FIG. 10d) for ski substitute. In addition, biochemical parameters as well as up regulation of prohealing markers such as hydroxyproline, hexosamine and study on inflammatory markers like TNF-α, IL-1α, C3a proved its efficacy.

### Results:

It was observed that DNA and protein content increased to a significant level in the rats treated with foam-based scaffold in comparison to the sham group.

Quantification of Hexosamine and hydroxyl proline markers is useful to understand the amount of collagen formed. An increase in collagen formation was observed in the foam-based scaffold treated wounds as compared with the reference group. Second degree burn wound lead to a significant tissue damage resulting in upregulation of various pro-inflammatory cytokines. Quantification of Tumor Necrosis Factor (TNF)-α day 0, 7, 14 and 21 and IL-1α level healthy, sham, treated and reference groups on day 0, 7, 14, 21 and 28 (FIG. 10e). TNF-α. activates keratinocytes and macrophages to produce Inflammatory mediators. The start of the inflammation activity after immediate cause of wound, helped in the release of the MMPs by TNF-α. MMPs help in remodeling the extracellular matrix by degrading the damaged matrix, so that formation of collagen and reepithelization. TNF-α level secretion was found to be significantly increased in sham group on 7 and 14 days in comparison to both treated and reference group, while TNF-α level was found to be very low in both treated and reference group on day 7 day 14 and day 21 while it was not detectable on day 28 in all the groups.

### IL-1^{α} Assay:

IL-1^{α} is also known as fibroblast activating factor (FAF). It is constitutively produced by epithelial cells and present in substantial amount in healthy human epidermis. It induces collagen synthesis, but excessive fibroblast formation leads to hypertrophic scar formation. IL-1α level was found to decrease till 14 days in both treated and reference group, while it reaches a level of the healthy rat at day 28 in both treated and reference group compared to sham group and it indicates the development of fibroblasts for the collagen synthesis on the wound bed which plays an effective role in the healing of partial-thickness—second degree burn wounds, FIG. 10f.

### C3a Assay:

C3a: Complement system is a vital part of the host innate immune system. A controlled expression of C3a helps in wound Healing but a significant rise in C3a can cause detrimental effect on burn wounds. An increase in the expression of C3a was found in all the three groups on day 7 while its expression reaches the level of healthy rat in the treated group and reference group on day 28 whereas sham group showed increased secretion on day 28 due to absence of scaffold to treat the wound in the given time, FIG. 10g).

A histopathology score card of treated and reference group is provided in (FIG. 10d). From the histopathological analysis as mentioned in the score card, as expected acute inflammation was observed only on day 7 in both skin substitute (treated group) and reference group (severe response on day 7). Mild to negative response of chronic inflammation was observed till day 28 in treated and reference group. Formation of edema, exudates and granular tissue formation was not observed on day 14, 21 and 28 in both treated group and reference group. Epithelization, collagen and capillary formation was well observed in both the treated and reference group till day 28. Skin was totally healed in case of both treated and reference groups on 28 days.

### Acute Systemic Toxicity Test of ‘Artificial Skin Substitute’ in Wistar Rats by Intravenous and Intraperitoneal Route

The objective of this study was to evaluate assess acute systemic toxicity potential of polar (normal saline) and non-polar (sesame oil) extracts of the test item ‘Artificial Skin Substitute’, when administered as a single dose through intravenous and Intraperitoneal route to Wistar rat respectively, as described in ISO 10993-11: Biological Evaluation of Medical Devices—Part 11: Tests for Systemic toxicity, 2017. The study was carried out in twenty rats. All rats received polar and non-polar test extracts and respective vehicle controls. Test item was cut into small pieces and representative parts were used for preparation of extracts. The test item was incubated along with polar and non-polar extracting media at the ratio of 3 cm2/mL in glass bottles kept in shaking incubator and maintained at 37° C. for 72 h. The respective extracting media alone served as the vehicle control and were treated in the same manner as the test item extract. No abnormal clinical signs or any mortality was recorded in any of the treated animal. Normal gain in body weight was recorded on Day 2, Day 3 and Day 7 compared to Day 1. No abnormality was detected in any of the animal during detailed clinical examination performed on Day 3 and Day 7. No gross pathological changes were observed in any of the treated animal. Under the experimental conditions of this study, polar (Normal Saline extracts) and non-polar extracts (oil Extracts) of test item “Artificial Skin Substitute” was found to be safe and no systemic toxicity was observed.

### Intracutaneous Reactivity Test of Artificial Skin Substitute in New Zealand White Rabbits

The objective of this study was to evaluate the possible local tissue reaction of polar (normal saline) and non-polar (sesame oil) extracts of the test item ‘Artificial Skin Substitute’, when injected intracutaneously to New Zealand white rabbits as described in ISO 10993-10: Biological Evaluation of Medical Devices—Part 10: Tests for Irritation and skin Sensitization: section 6.4; Animal Intracutaneous (intradermal) reactivity test, 2010. The study was carried out in three rabbits. All rabbits received polar and nonpolar test extracts and respective vehicle controls. The test item was cut into small pieces and used for preparation of extracts. The test item along with polar and non-polar extracting media at the ratio of 0.2 g/ml in bottles were kept in shaker incubator and maintained at 37° C. for 72 h. without agitation. The extracts were injected intracutaneously at a dose of 0.2 ml per site, at 5 sites on left dorsal side of two rabbits. Similarly, at 5 sites on the other side of spinal column of two rabbits the corresponding vehicle control was injected. The injected sites were observed immediately and approximately at 24, 48 and 72 h for any gross evidence of tissue reactions such as erythema and edema. During the course of study all the animals were observed for mortality/morbidity and body weight.

No mortality/morbidity or any abnormal clinical signs in any of the animal was observed. Normal gain in body weight on Day 4 compared to Day 1 was seen. No local tissue reaction (erythema and edema) was found at the site of injection. The difference between the test extract and the respective vehicle control mean scores was found to be zero. Under the test conditions the skin substitute was found to be nonirritant

Although the subject matter has been described in considerable detail with reference to certain preferred embodiments thereof, other embodiments are possible. As such, the spirit and scope of the subject matter should not be limited to the description of the preferred embodiment contained therein.

The novel artificial foam-based skin substitute scaffold of the present invention addresses the problems in the prior art by providing a technically advanced solution. The various advantages inter alia are listed below:

### Advantages of the Claimed Invention

Biocompatible;
Biodegradable;
Bioabsorbable;
Prevents scar formation;
Non-immunogenic;
Non-irritant—does not result in erythema or edema
No exudate
Easy applicability;
Cost effective

1. An acellular foam based artificial skin substitute comprising:
a) a cross-linked porous upper layer, and
b) a non-cross-linked non-porous bottom layer
characterized in that the said acellular foam based artificial skin substitute comprises a biopolymer in the range of 95 to 99% and a bioactive component in the range of 1 to 5% based on the weight of biopolymer.

2. The acellular artificial skin substitute as claimed in claim 1, wherein the artificial skin substitute is a scaffold of 2-4 mm proportion.

3. The acellular artificial skin substitute as claimed in claim 1, wherein the porous upper layer is crosslinked with a crosslinker selected from the group consisting of EDC, glutaraldehyde, natural polyphenolic crosslinkers like caffeic acid, genipin and tannic acid.

4. The acellular artificial skin substitute as claimed in claim 3, wherein the concentration of the said crosslinker ranges between 1-10 mM.

5. The acellular artificial skin substitute as claimed in claim 1, wherein said biopolymer is selected from gelatin, elastin, collagen, pectin, laminin, fibronectin and mixtures thereof.

6. The acellular artificial skin substitute as claimed in claim 1, wherein the said bioactive component is selected from hyaluronic acid, chondroitin sulfate, Dermatan sulfate and keratin sulfate and mixtures thereof.

7. The acellular artificial skin substitute as claimed in claim 1, wherein the pore size of the said acellular artificial skin substitute is in the range of about 60 to about 300 μm.

8. A process for preparing of acellular foam based artificial skin substitute comprising the steps of:
i. obtaining a solubilized biopolymer by dissolving said biopolymer in water,
ii. adding bioactive components to the solubilized biopolymer and stirring continuously at appropriate temperature and for appropriate time to obtain a dissolved composite viscous solution,
iii. placing the dissolved composite viscous solution under foam maker,
iv. casting the composite viscous solution as obtained in step (iv) on a petri-plate and distributing the same homogenously for uniform thickness,
v. Adding the crosslinker in a specific manner to selectively crosslink the composite viscous solution such that only the upper layer gets crosslinked,
vi. Obtaining the selectively crosslinked acellular artificial skin substitute having a cross-linked porous upper layer, and a non-cross-linked non-porous bottom layer,
vii. Removing excess cross linker by repeated washing,
viii. pre-freezing the artificial skin substitute at −40 to −30° C. for 2-3 hours, and
ix. lyophilizing the acellular artificial skin substitute.

9. The process as claimed in claim 8, wherein the solubilized biopolymer is obtained by dissolving in water at 40-45° C. for 10-30 minutes.

10. The process as claimed in claim 8, wherein the bioactive components are added to the solubilized bio polymer obtained in step (i) and stirred continuously at 40-45° C. for 1-4 hours to obtain a dissolved composite viscous solution.

11. The process as claimed in claim 8, wherein the dissolved composite viscous solution of step (ii) is put under foam maker at 1200 to 15,000 rpm for 1-5 minutes.

12. The process as claimed in claim 8, wherein the viscous solution obtained in step (iii) is cast on the petri plate by homogeneous distribution and keeping the petri plate undisturbed for 30 minutes at 25-30° C.

13. The process as claimed in claim 8, wherein the homogenously cast viscous solution is selectively cross-linked by adding the cross-linker in a specific manner for 10-20 minutes at 4° C. to 8° C. to obtain the acellular skin substitute having a cross-linked porous upper layer, and a non-cross-linked non-porous bottom layer with uniform thickness.

14. The process as claimed in claim 8, wherein the artificial skin substitute prepared in step (vi) is kept for pre-freezing at −40 to −30° C. for 2-3 hours.

15. The process as claimed in claim 8, wherein the acellular skin substitute obtained in step (vii) is lyophilized step-wise as follows:
Step 1: at −30° C. for 1-3 hours
Step 2: at −20° C. for 2-4 hours
Step 3: at −10° C. for 2-3 hours
Step 4: at 1° C. for 3-4 hours
Step 5: at 5° C. for 2-3 hours
Step 6: at 10° C. for 2-3 hours
Step 7: at 20° C. for 3-4 hours
wherein all the lyophilization steps are carried out at a vacuum of 0.06 Mbar pressure.

16. A skin graft comprising the acellular artificial skin substitute as claimed in claim 1.

17. A wound dressing comprising the acellular artificial skin substitute as claimed in claim 1.

18. A kit comprising of the acellular foam based artificial skin substitute as claimed in claim 1, optionally along with instructions for its use.

19. A method for treating wound related infections and/or promoting wound healing in a subject in need of such treatment comprising applying the wound dressing as claimed in claim 17 to the affected area and/or part of the subject.

20. The method of claim 19, wherein the wound is associated with a burn or trauma.
